(19) United States
(12) Patent Application Publication
(10) Pub. No.: US 2022/0241000 A1
(43) Pub. Date: Aug. 4, 2022

(54) ELECTROCAUTERY APPARATUS AND METHOD FEATURING ULTRASOUND GUIDANCE

(71) Applicant: Little Engine LLC, Greenwich, CT (US)

(72) Inventors: Stacey Clarfield Newman, Greenwich, CT (US); Fredric Newman, Greenwich, CT (US)

(21) Appl. No.: 17/589,043

(22) Filed: Jan. 31, 2022

Related U.S. Application Data
(60) Provisional application No. 63/144,297, filed on Feb. 1, 2021.

(51) Int. Cl. A61B 18/08; A61B 18/10; A61B 34/10

(52) U.S. Cl. CPC A61B 18/085; A61B 2018/00994; A61B 34/10; A61B 18/10

(57) ABSTRACT

The present invention includes electrocautery devices and methods for surgery. The present invention includes an electrocautery device (or other surgical cutting and dissecting tool; harmonic scalpel; scissors capable of cutting, dissecting and obtaining hemostasis) comprising: (a) a handle portion; (b) an electrocautery unit integrated into said handle portion, and comprising a surgical electrode extending from said distal end thereof; (c) an ultrasonic transponder integrated into said handle portion and extending from said distal end thereof and disposed adjacent to said surgical electrode and so as to be adapted to detect differences in anatomical tissue types distally of said surgical electrode; (d) a source of current to said surgical electrode; (e) a feedback module adapted to either (1) signal the user of the electrocautery device or (2) change or interrupt the current provided by said source of current; and (f) data transmission module for transmitting data from said ultrasonic transponder, and for transmitting data to said feedback module. The present invention may be adapted to be used in conjunction with other types of scalpels, scissors, etc.; i.e., cutting and dissecting devices.

### RELATED APPLICATION DATA

This application claims the priority benefit of U.S. Provisional Application Ser. No. 63/144,297, filed Feb. 1, 2021, which is hereby incorporated in its entirety herein by reference.

### FIELD OF THE INVENTION

The present invention relates to devices and techniques that may be used in electrocautery scalpel guidance and feedback systems, and in similar surgical, therapeutic and testing techniques. The present invention may also be adapted to be used for the guidance and feedback in other types of scalpels, scissors, etc.; i.e., cutting and dissecting devices.

### BACKGROUND OF THE INVENTION

There are a wide variety of surgical operations that involve tissue, organ removal and/or repositioning and rearrangement.

Electrocautery scalpel systems provide the advantage of immediate and effective tissue cutting as well as the advantage of hemostasis that is particularly important in limited access procedures.

An area of particular interest is in those applications involving surgery around tissue structures that are near nerve tissues that present a risk of severe injury if injured or severed. These tissues include major organ tissues, veins, arteries, nerves, and glands.

Breast-area procedures may also result in blood vessel damage that can result in undetected bleeding. Where resection is called for (such as in breast cancer procedures), incomplete resection may lead to tumor recurrence and metastatic cancer.

Although improvements have been made to surgical devices and techniques, specifically to those that use electrocautery knives, there remains a need for improved guidance and feedback systems to avoid nerve damage in the various surgical operations where electrocautery knives and other dissecting devices may be beneficially applied.

There also remains a significant need to improve nerve avoidance in surgical procedures throughout the body, particularly in and around neurologically complex anatomy such as breast, pelvic, and oral maxillofacial areas.

Similarly, there remains a need for improved guidance and feedback systems that may be used in other types of scalpels, scissors, etc.; i.e., generally cutting and dissecting devices.

### SUMMARY OF THE INVENTION

The present invention includes surgical tools designed to reliably achieve a new level of safety for even the finest operators. These may include electrocautery scalpel systems as well as other types of scalpels, scissors, etc.; i.e., cutting and dissecting tools and devices for cutting, dissecting and/or obtaining hemostasis, such as harmonic scalpels, etc., generally referred to herein as surgical cutting devices. The present invention includes a medical scalpel device adapted to avoid injury to nerves, such as the long thoracic nerve by providing a surgical cutting tool that can produce real-time, in situ feedback to the surgeon regarding nerve location to avoid nerve injury, such as might otherwise occur through surgical (iatrogenic) injury from scalpels and other tools.

Some embodiments of the present invention may include a medical scalpel with tissue mapping and proximity feedback with optional automatic shut-off.

Further optional embodiments of the present invention may include augmented reality functions, additional functionality that enable dissection, cutting, irrigation and cauterization of tissue, the evacuation of gasses and fluids from the procedure site, and blood vessel avoidance.

The devices and systems of the present invention may be advantageously used in any surgical procedure throughout the body (i.e., the face, body or extremities), particularly in high risk surgical procedures, such as those procedures around and involving the prostate, breast and facial tissues. Such surgical procedures are typically affected by such crucial factors as the intrinsic risk due to the location of the procedure, the visibility in the field, and the operator's individual skills, perceptions and abilities.

It is an object of the present invention to provide a safe medical scalpel device. The object is achieved by a medical scalpel device and method according to the independent claims. Other more specific embodiments of the invention are given in the dependent claims.

In the context of this specification the term distal refers to the direction pointing towards the patient during a surgical procedure, while the term proximal refers to the opposite direction pointing away from the patient. In the context of this specification the term proximal end refers to the end of the device closer to the patient during a surgical procedure, while the term distal end refers to the opposite end nearest the operator of the device; i.e., furthest from the patient.

The electrocautery devices and systems of the present invention may be considered improvements upon electrocautery devices and systems such as those commercially available from Bovie Medical Corporation of Clearwater, Fla., and Symmetry Surgical of Antioch, Tenn., USA. Such devices include those described in the following US patents and published applications, all of which references are hereby incorporated herein by reference:

| Pat. No. | Title |
|---|---|
| 10,064,675 | Multi-mode electrosurgical apparatus |
| 9,770,285 | System and method for identifying and controlling an |
| | electrosurgical apparatus |
| 9,770,281 | Electrosurgical apparatus with retractable blade |
| 9,763,724 | Systems and methods of discriminating between argon |
| | and helium gases for enhanced safety of medical devices |
| 9,681,907 | Electrosurgical apparatus to generate a dual plasma |
| | stream and method thereof |
| 9,649,143 | Electrosurgical system to generate a pulsed plasma |
| | stream and method thereof |
| 9,572,621 | Surgical jaws for sealing tissue |
| 9,326,810 | Multi-button electrosurgical apparatus |
| 9,144,453 | Multi-mode electrosurgical apparatus |
| 9,060,765 | Electrosurgical apparatus with retractable blade |
| 8,998,899 | Multi-button electrosurgical apparatus |
| 8,979,834 | Laparoscopic electrosurgical electrical leakage detection |
| 8,795,265 | Electrosurgical apparatus to generate a dual plasma |
| | stream and method thereof |
| 8,696,663 | Electromechanical polyp snare |
| 8,628,524 | Return electrode detection and monitoring system and |
| | method thereof |
| 8,409,190 | Electrosurgical device to generate a plasma stream |
| 8,226,640 | Laparoscopic electrosurgical electrical leakage detection |
| 8,114,181 | Reflux trap device |
| 8,100,897 | Laparoscopic electrosurgical electrical leakage detection |
| 8,057,468 | Method to generate a plasma stream for performing |
| | electrosurgery |
| 7,632,270 | Multi-mode surgical instrument |
| Published | |
| Application No. | Title |
| 20140018795 | Multi-Button Electrosurgical Apparatus |
| 20140005665 | Systems and Methods of Discriminating Between Argon |
| | and Helium Gases for Enhanced Safety of Medical |
| | Devices |
| 20130237982 | Multi-Mode Electrosurgical Apparatus |
| 20120116397 | Electrosurgical Apparatus with Retractable Blade |
| 20120065635 | Electrosurgical Device to Generate A Plasma Stream |
| 20110221463 | Scanning Cannula |
| 20100331835 | Return Electrode Detection and Monitoring System and |
| | Method Thereof |
| 20100305564 | Surgical Jaws for Sealing Tissue |

The electrocautery devices and systems of the present invention may incorporate a three-dimensional spatial detection system that communicates between the computer and the device handle, and which provides associated tactile feedback in response to movement of the device handle with respect to the imaged surgical site in or to warn the operator of positioning of the electrocautery device in a proximity to tissue whose cutting or damage is to be avoided. Such a three-dimensional spatial detection system and associated tactile feedback systems are commercially available from Nintendo of America of Redmond, Wash., and which are described in one or more of the following US patents the entire disclosure of all of which is hereby incorporated by reference: U.S. Pat. Nos. 7,568,975; 7,905,781; 8,267,780; 8,337,304; 8,384,770; 8,442,436; 8,608,392; 8,628,419; 8,636,595; 8,641,527; 8,647,205; 8,768,255; 8,848,100; 8,851,997; 8,858,337; 8,894,486; 8,917,985; 8,951,122; 9,174,126; 9,174,129; 9,180,376; 9,320,972; 9,367,147; 9,387,404; 9,457,267; 9,457,268; 9,510,472; 9,724,601; 9,751,008; 9,757,647; 9,776,081; 9,776,082; 9,782,671; 9,895,606; 10,074,269; 10,092,832; 10,139,865; 10,220,309; 10,258,879; 10,354,519; and 10,661,160.

The present invention may be summarized as follows:

### SUMMARY OF THE ELEMENTS

There are several optional arrangements of the present invention that are described in its many embodiments.

In general terms, the electrocautery device of the present invention comprises: (a) a handle portion; (b) an electrocautery unit integrated into the handle portion, and comprising a surgical electrode extending from the distal end thereof; (c) an ultrasonic transponder integrated into the handle portion and extending from the distal end thereof and disposed adjacent to the surgical electrode and so as to be adapted to detect differences in anatomical tissue types proximally of the surgical electrode; (d) a source of current to the surgical electrode, (e) a feedback module adapted to either (1) signal the user of the electrocautery device or (2) change or interrupt the current provided by the source of current, and (f) data transmission module for transmitting data from the ultrasonic transponder, and for transmitting data to the feedback module. In the context of this specification, the term distal refers to the direction or vector toward the patient and the term proximal refers to the direction or vector toward the operator.

Although the device of the present invention may be described in the context of an electrocautery scalpel system, it will be appreciated that the present invention may be adapted to be used in conjunction with other types of scalpels, scissors, etc.; i.e., cutting and dissecting devices.

The electrocautery device of the present invention may optionally include (g) an accelerometer and a short-range wireless data transmission module adapted to transmit position and acceleration data from the electrocautery unit, as well as electrocautery unit status data. The data transmission module also contains a microprocessor and RAM/ROM memory for managing the short-range wireless interface and converting voltage data from the accelerometer into digitized data.

The electrocautery device of the present invention may be an element of a system that includes a microprocessor and display adapted to display the surgical site and further incorporate position and acceleration data from the electrocautery unit.

The electrocautery device and system of the present invention may optionally include (h) a three-dimensional spatial detection system and associated tactile feedback system contained within the electrocautery device and the associated computer system. The electrocautery device additionally comprises a three-dimensional spatial data module adapted to transmit three-dimensional spatial data to a computer system to provide visual or tactile feedback to the electrocautery device handle, and to turn off the electrocautery device in the event tissue to be preserved is detected through coordination with other imaging techniques and modalities, such as those described herein.

The electrocautery device and system of the present invention may optionally include: (i) a computer adapted to accept data from the ultrasonic transponder through the data transmission module and to process the data and signal the feedback module so as to cause the feedback module to either (1) signal the user of the electrocautery device (or any other cutting and dissecting device) or (2) change or interrupt the current provided by the source of current.

### Methods

The present invention also includes a method of using an electrocautery device. The present invention in general terms comprises a method for conducting a surgical procedure using an electrocautery, the method comprising: (1) extending into the surgical region a device comprising: (a) a handle portion; (b) an electrocautery unit integrated into the handle portion, and comprising a surgical electrode extending from the distal end thereof; (c) an ultrasonic transponder integrated into the handle portion and extending from the distal end thereof and disposed adjacent to the surgical electrode and so as to be adapted to detect differences in anatomical tissue types distally of the surgical electrode; (d) a source of current to the surgical electrode, (e) a feedback module adapted to either (1) signal the user of the electrocautery device or (2) change the current provided by the source of current, and (f) data transmission module for transmitting data from the ultrasonic transponder, and for transmitting data to the feedback module; and (2) cutting tissue within the surgical region while the cutting is guided and/or controlled through the feedback module by either (1) signaling the user of the electrocautery device or (2) changing the current provided by the source of current.

The method may also include having the electrocautery device of the present invention include (g) an accelerometer and a short-range wireless data transmission module adapted to transmit position and acceleration data from the electrocautery unit, as well as electrocautery unit status data, and wherein the cutting is guided and/or controlled through the feedback module by providing relative position data to the user or otherwise automatically changing the current provided by the source of current.

The method may also include having the electrocautery device and system of the present invention include (h) a three-dimensional spatial detection system and associated tactile feedback system contained within the electrocautery device and the associated computer system, with the electrocautery device additionally comprising a three-dimensional spatial data module adapted to transmit three-dimensional spatial data to a computer system to provide visual or tactile feedback to the electrocautery device handle, and to turn off the electrocautery device in the event tissue to be preserved is detected through coordination with other imaging techniques and modalities, such as those described herein, and wherein the cutting is guided and/or controlled through that feedback module by providing relative position data to the user or otherwise automatically changing the current provided by the source of current.

Although the methods of the present invention may be described in the context of an electrocautery scalpel system, it will be appreciated that the methods of the present invention may be carried out using other types of scalpels, scissors, etc.; i.e., cutting and dissecting devices into which such feedback and guidance elements have been incorporated.

The foregoing and other objects, features, and advantages of this invention will become more readily apparent from the following detailed description of a preferred embodiment which proceeds with reference to the accompanying drawings, wherein one embodiment of the invention is shown and described, simply by way of illustration of the best mode contemplated of carrying out the invention.

As will be realized, the invention is capable of other and different embodiments, and its several details are capable of modifications in various obvious respects, all without departing from the invention. Accordingly, the drawings and description are to be regarded as illustrative in nature, and not as restrictive. It will also be appreciated that the detailed description represents one embodiment of the invention, and that individual steps of the process of the invention may be practiced independently so as to achieve similar results.

### BRIEF DESCRIPTION OF THE DRAWINGS

FIG. 1 is a schematic view of an electrocautery device connected to a computer with display, in accordance with one embodiment of the present invention.

FIG. 2 is a lateral elevation view of an electrocautery device in accordance with one embodiment of the present invention showing the incorporation of an electrocautery blade.

FIG. 3 is a lateral, partially exploded elevation view of an electrocautery device in accordance with one embodiment of the present invention showing the incorporation of an electrocautery blade.

FIG. 4 is a lateral, partially exploded elevation view of an electrocautery device in accordance with another embodiment of the present invention showing the incorporation of an electrocautery attachment.

FIG. 5 is a lateral elevation view of an electrocautery device in accordance with another embodiment of the present invention showing the incorporation of an electrocautery attachment.

FIG. 6 is a detailed proximal end perspective view of an electrocautery device in accordance with another embodiment of the present invention showing the incorporation of an electrocautery blade.

FIG. 7 is a top plan view of an electrocautery device in accordance with one embodiment of the present invention showing the incorporation of an electrocautery blade.

FIG. 8 is a top plan, partially exploded elevation view of an electrocautery device in accordance with one embodiment of the present invention showing the incorporation of an electrocautery blade.

FIG. 9 is a top plan, partially exploded elevation view of an electrocautery device in accordance with another embodiment of the present invention showing the incorporation of an electrocautery attachment.

FIG. 10 is a top plan view of an electrocautery device in accordance with another embodiment of the present invention showing the incorporation of an electrocautery attachment.

FIG. 11 is a lateral elevation view of an electrocautery device in accordance with another embodiment of the present invention showing the incorporation of the ultrasonic probe.

FIG. 12 is an overhead view of a surgical environment showing use of an electrocautery device and system in accordance with one embodiment of the present invention.

FIG. 13 is schematic of the device sensing, signal and microprocessor logic and feedback logic used in an electrocautery device and system in accordance with one embodiment of the present invention.

FIG. 14 shows a patient during delivery of a biomarker to better elucidate the surgical environment to directly locate anatomical features in accordance with one embodiment of the present invention.

FIG. 15 shows a logic diagram describing an example of the computer logic that may be carried out in accordance with one embodiment of the present invention.

### DETAILED DESCRIPTION OF THE PREFERRED EMBODIMENTS

In accordance with the foregoing summary, the following provides a detailed description of the preferred embodiments, which are presently considered to be the respective best modes thereof.

As used herein the distal end refers to the working end or patient end, while the proximal end refers to the operator end or actuator end from which the device of the present invention may be operated. The side opposite the bottom side is referred to as the top side or dorsal aspect. The right side is the side on the right hand when looking from the operator end, end-on. Conversely, the left side is the side on the left hand when looking from the operator end, end-on.

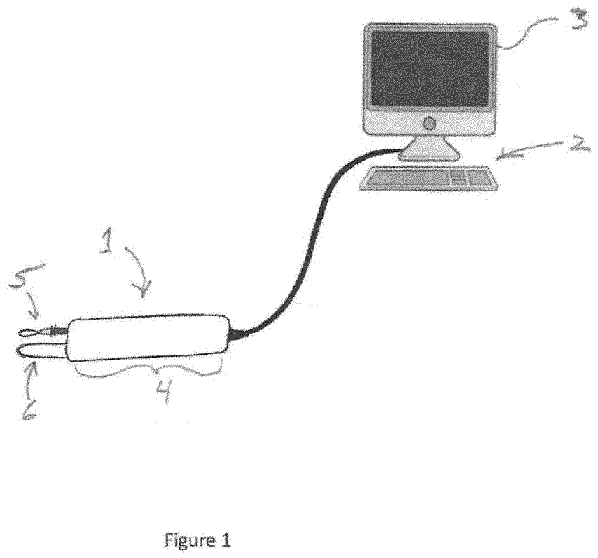

FIG. 1 is a schematic view of an electrocautery device 1 connected to a computer 2 with display 3, in accordance with one embodiment of the present invention.

FIG. 1 shows a handle portion 4 that can be wired or wireless for both power and data transmission, such as Bluetooth or WiFi 2.4 GHz wireless data transmission. The electrocautery device 1 may be battery or wired power, depending on the application power requirements and industry feedback. The displayed embodiment shows how a commercially available electrocautery unit or pen 5 that may be fit into the handle portion 4. The electrocautery unit 5 is integrated into the handle portion 4, and comprises a surgical electrode extending from the distal end thereof. The ultrasonic transponder 6 is likewise integrated into the handle portion 4.

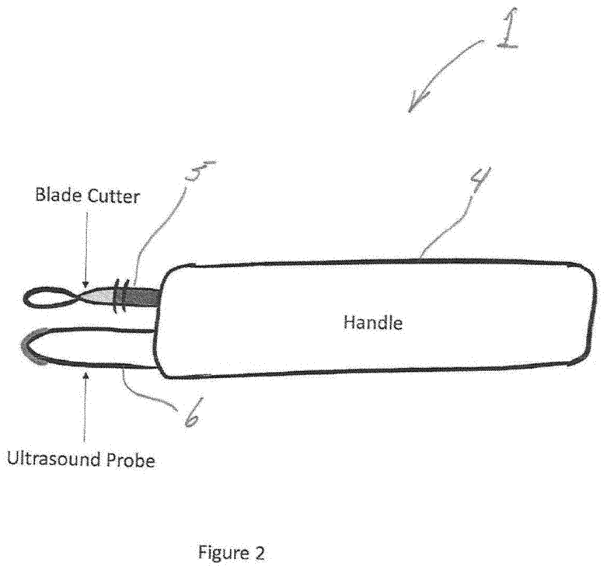

FIG. 2 is a lateral elevation view of an electrocautery device in accordance with one embodiment of the present invention showing the incorporation of an electrocautery blade, and wherein like numerals refer to the elements referenced above.

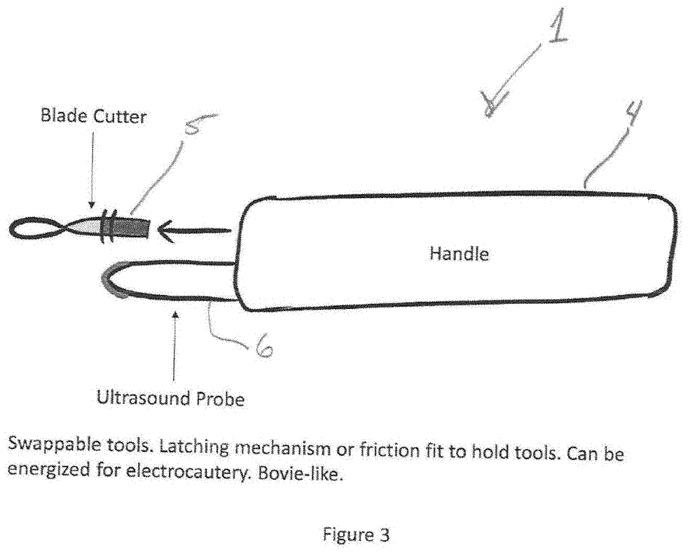

FIG. 3 is a lateral, partially exploded elevation view of an electrocautery device in accordance with one embodiment of the present invention showing the incorporation of an electrocautery blade, and wherein like numerals refer to the elements referenced above. This Figure shows how electrocautery unit 5 may be removed from handle 4.

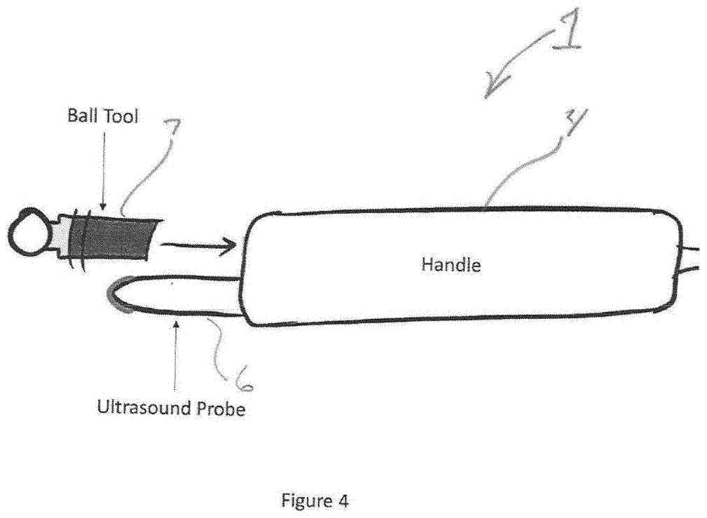

FIG. 4 is a lateral, partially exploded elevation view of an electrocautery device in accordance with another embodiment of the present invention showing the incorporation of the electrocautery attachment 7, and wherein otherwise like numerals refer to the elements referenced above. This Figure shows how electrocautery attachment 7 may be removed from handle 4 following removal of electrocautery unit 5, and vice versa.

The electrocautery attachment 7 may include electrosurgery electrodes and pencils such as those commercially available from Bovie Medical Corporation (commonly referred to as “Bovie tips” regardless of commercial source), or equivalent stainless steel electrodes.

Examples may include the Resistick II™ line of coated electrodes, including coated blades, balls, or needles made for many different uses, such as those bearing a polytetrafluoroethylene (PTFE) coating.

The electrocautery attachment 7 may include suction coagulators that can be used with hand or foot control, that may offer suction with or without coagulation at the same time, such as those in diameter sizes of 08, 10, and 12 in French Size.

The electrocautery attachment 7 may include standard electrocautery pencils that may be controlled with a button, rocker or footswitch. They are also available in models that can be either reused or disposable depending on the needs of the surgeon or facility. Common accessories for these pencils may include a multitude of disposable and reusable electrodes, disposable scratch pads and pencil holsters.

Two other common alternative electrosurgery attachments are disposable and reusable loops that may be made of tungsten wire with a standard 2.3 mm shaft for use in most electrosurgical pencils.

The alternative electrocautery attachments may include high temperature, low temperature, and change-a-tip battery operated cauteries.

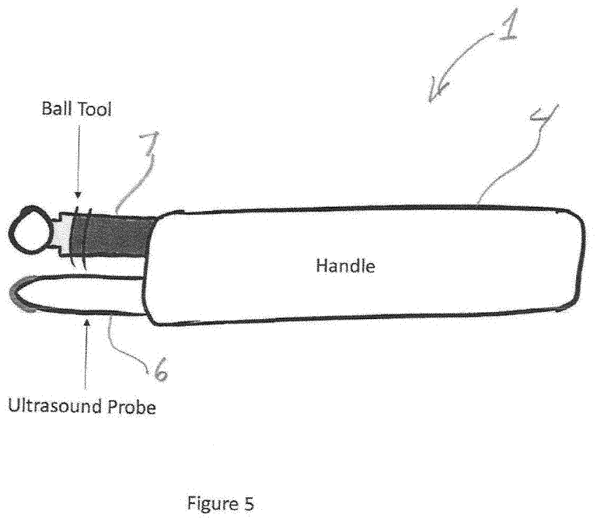

FIG. 5 is a lateral elevation view of an electrocautery device in accordance with another embodiment of the present invention showing the incorporation of an electrocautery attachment, and wherein like numerals refer to the elements referenced above. This Figure shows electrocautery attachment 7 fully integrated into handle 4.

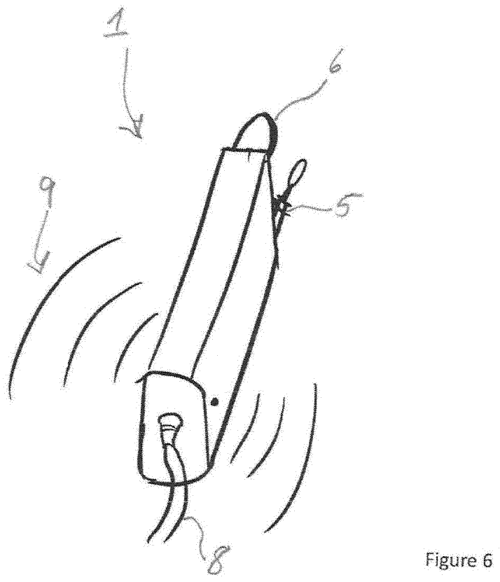

FIG. 6 is a detailed distal end perspective view of an electrocautery device 1 in accordance with another embodiment of the present invention showing the incorporation of an electrocautery unit 5, and wherein otherwise like numerals refer to the elements referenced above. This Figure shows that handle portion 4 may be wired through wire 8 or wireless through short-range wireless transmission 9, and that either or both may be utilized for power and data transmission.

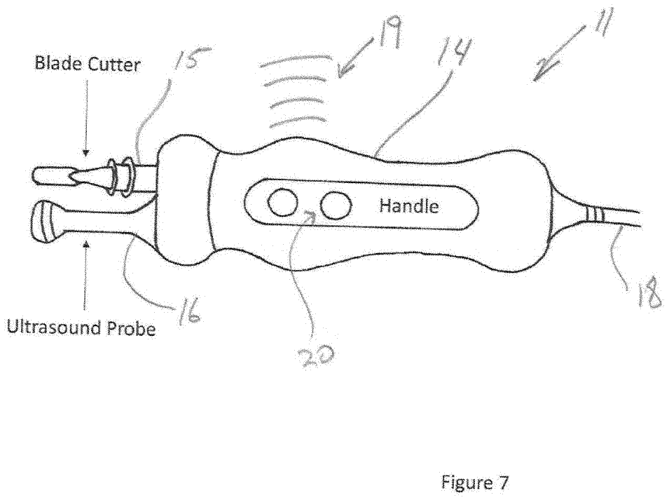

FIG. 7 is a top plan view of an electrocautery device 11 in accordance with another embodiment of the present invention showing the incorporation of an electrocautery blade, and that may be attached or wirelessly associated with a computer such as computer 2 with display 3, as shown in FIG. 1.

FIG. 7 shows a handle portion 14 that can be wired or wireless for both power and data transmission, such as Bluetooth or WiFi 2.4 GHz wireless data transmission. The electrocautery device 11 may be battery or wired power, depending on the application power requirements and industry feedback. The displayed embodiment shows how a commercially available electrocautery unit or pen 15 that may be fit into the handle portion 14. The electrocautery unit 15 is integrated into the handle portion 4, and comprises a surgical electrode extending from the distal end thereof. The ultrasonic transponder 16 is likewise integrated into the handle portion 14. This Figure shows that handle portion 14 may be wired through wire 18 or wireless through short-range wireless transmission 19, and that either or both may be utilized for power and data transmission. Also shown are device controls 20.

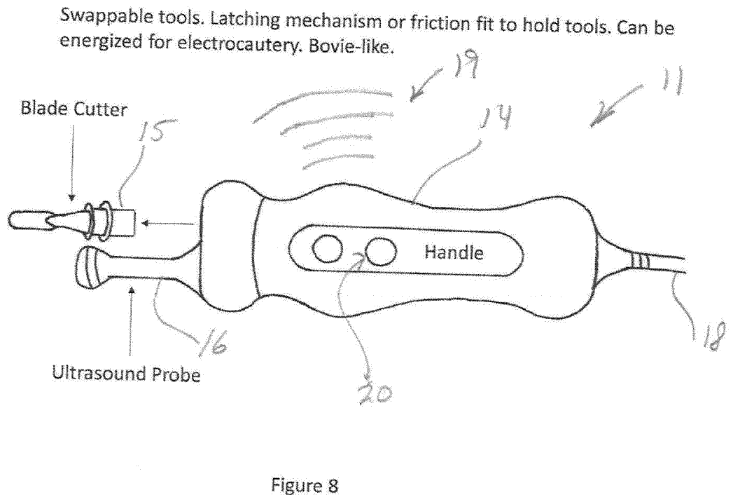

FIG. 8 is a top plan, partially exploded elevation view of an electrocautery device 11 in accordance with one embodiment of the present invention showing the incorporation of an electrocautery unit or pen 15, and wherein like numerals refer to the elements referenced above. This Figure shows how electrocautery unit or pen 15 may be removed from handle 14.

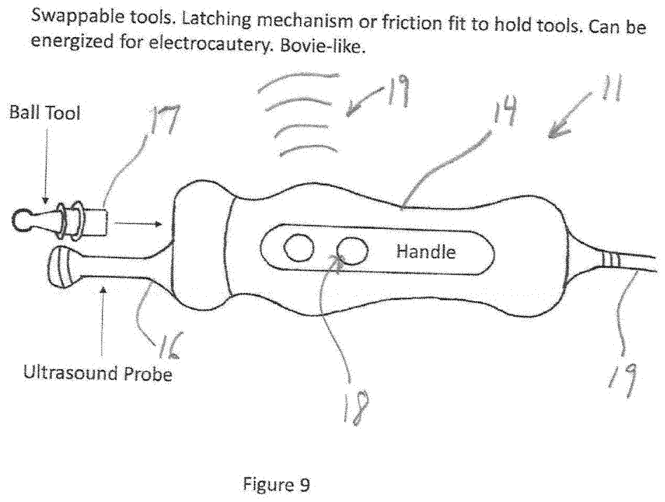

FIG. 9 is a top plan, partially exploded elevation view of an electrocautery device 11 in accordance with another embodiment of the present invention showing the removal of electrocautery attachment 17, and wherein otherwise like numerals refer to the elements referenced above.

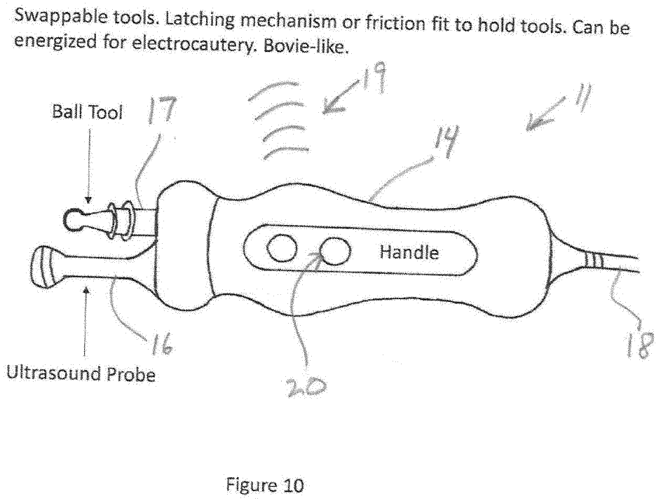

FIG. 10 is a top plan view of an electrocautery device 11 in accordance with another embodiment of the present invention showing the incorporation of electrocautery attachment 17, and wherein otherwise like numerals refer to the elements referenced above.

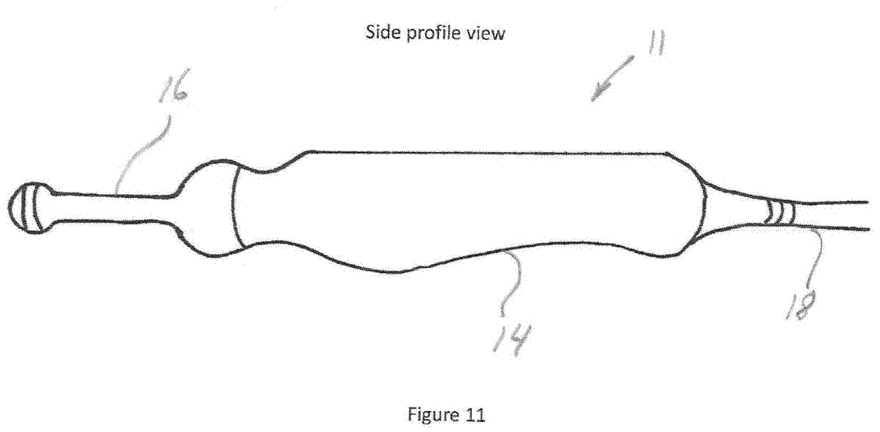

FIG. 11 is a lateral elevation view of an electrocautery device 11 in accordance with another embodiment of the present invention showing the incorporation of the ultrasonic probe 16, and wherein otherwise like numerals refer to the elements referenced above.

As to the procedure for using the device of the present invention and otherwise to practice its method, the following steps may be used:

In order to prepare and use the device of the present invention as exemplified by the embodiment shown in FIGS. 1-11, the following procedure may be used as described stepwise by reference to those Figures.

### Ultrasound

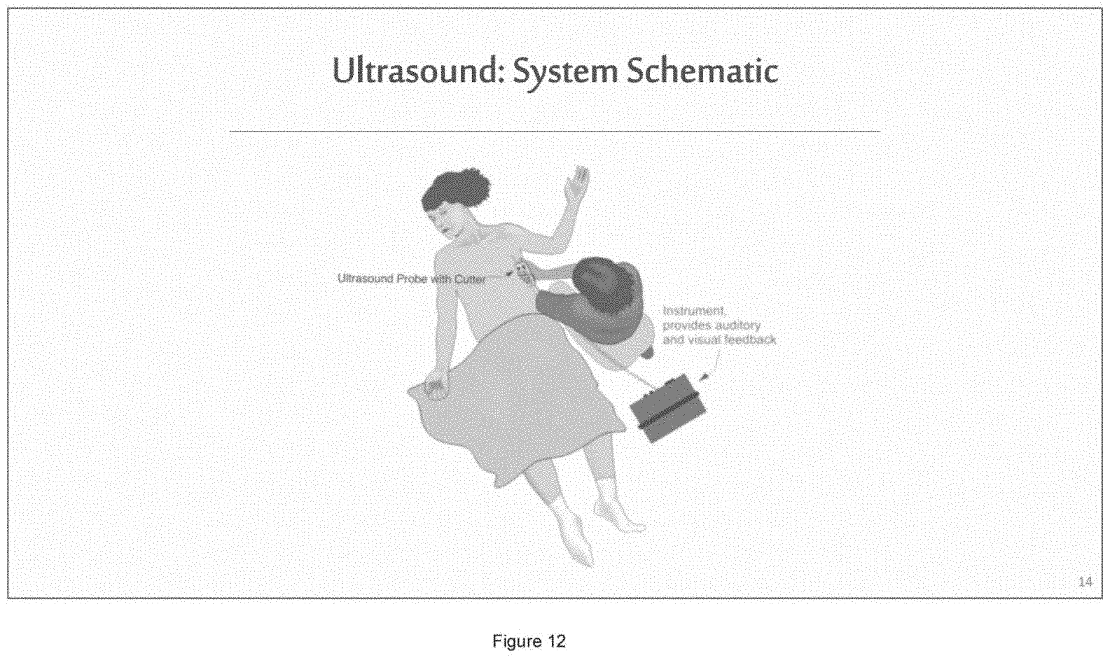

FIG. 12 is an overhead view of a surgical environment showing use of an electrocautery device and system in accordance with one embodiment of the present invention. The electrocautery device and system may be used to avoid injury to nerve tissue, such as the long thoracic nerve, by providing a surgical cutting tool that is capable of producing real-time, in situ feedback to the surgeon regarding nerve location to avoid injuring it. As one instructive example, this may be done by utilizing a direct approach whereby the long thoracic nerve is directly visualized as it is appreciably larger than surrounding nerves, facilitating differentiation.

By contrast, an indirect approach may be used whereby the nerve location can be deduced from location of arteries and veins, which are located alongside it in a tightly formed “bundle,” as axillary artery and vein have a distinct size relative to surrounding vessels as well as different anatomical position, either of these parameters may be used for facilitating differentiation.

The electrocautery device and system of the present invention may in some embodiments incorporate high-resolution ultrasound coupled with a cutting instrument to facilitate real-time detection of target tissue (vessel or nerve), permitting the surgeon to make a judgement during surgery based on tissue detection, and the electrocautery device and system may further be automated such that automatic shut-off occurs when within a certain proximity of target tissue is reached.

The electrocautery device and system of the present invention may be used in accordance with a stimulus probe to integrate with the electrocautery cutting tool.

In other embodiments of the present invention, as an alternative to or supplement of the use of high-resolution ultrasound coupled with a cutting instrument to facilitate real-time detection of target tissue (vessel or nerve), such embodiments of the present invention may include the use of a Doppler ultrasound imaging module that may be used to further detect and elucidate blood vessels within proximity of target tissue, including detecting blood flow, blood clots or blocked or narrowed blood vessels.

### Neuromonitoring

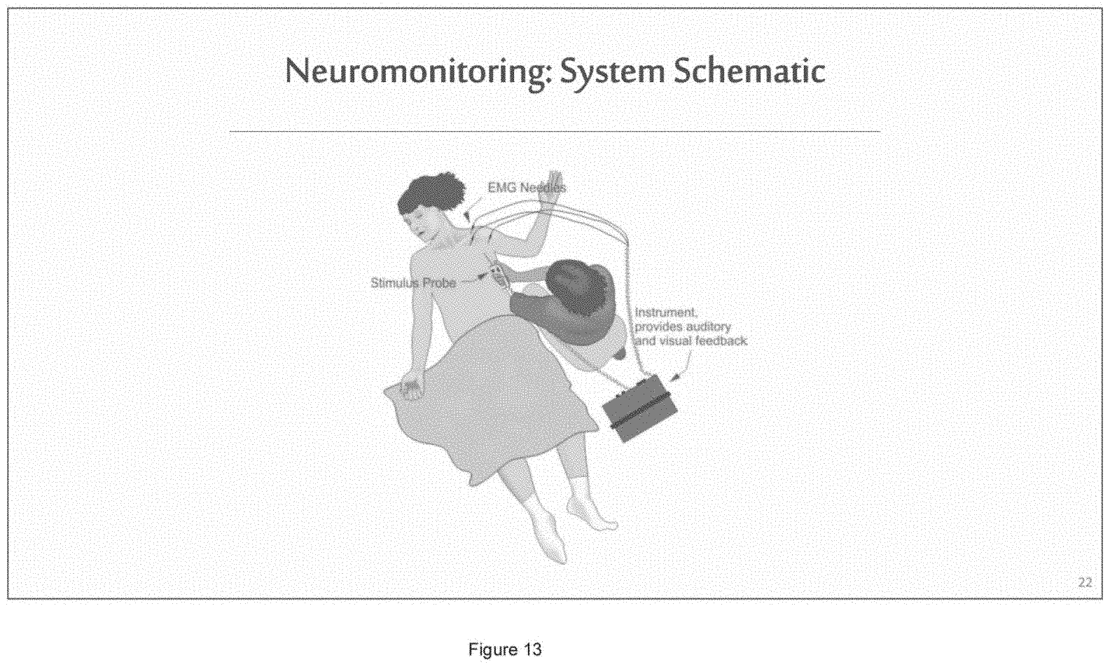

FIG. 13 is an overhead view of a surgical environment showing use of an electrocautery device and system in accordance with another embodiment of the present invention.

FIG. 13 shows neuromonitoring of a patient during delivery of an electrical stimulus to a target nerve with resultant nerve or muscle activity detected to provide feedback on quality of or proximity to neural pathways. Neuromonitoring may be used to monitor nerve pathways or muscle movement to directly locate anatomy or detect neural pathway degradation.

This embodiment of the electrocautery device and system of the present invention may detect muscle activity using needle probes in muscles (electromyography—EMG). Needles may be placed in easily located muscles that are enervated by the neural pathway of interest. Separate stimulus via other needles or movable probe may apply variable charge to area of the nerve. Proximity to the nerve is deduced from how much charge is needed to trigger muscle movement. This may be used for real-time location of nerves.

This embodiment of the present invention also permits sensory nerve pathways to be monitored directly. Needle probes may be placed at proximal sensory points for direct stimulation, and monitoring needle probes may be placed along known neural pathways for the sensory nerve signal as they ascend to and along the spinal cord. The neural pathway may be monitored for neural activity related to the stimulus, and signals are at a relatively very low level, so averaging over time is used to separate nerve signals from noise.

This embodiment of the present invention may be used in accordance with known electromyography techniques, as well as those used in common spine surgery to detect proximity to nerve root in the spinal cord area.

The electrocautery device and system of the present invention may be tuned to identify the long thoracic nerve with needle EMG.

The electrocautery device and system of the present invention may be tuned to determine basic reliable correlations between stimulus and response, and to determine pathways for monitoring of motor and sensory nerves.

In some embodiments to the electrocautery device and system of the present invention, the needle EMG signal and the electrocautery device are coordinated in order to permit the system to check for interference and artifacts between instruments, and to attenuate settings to optimize real time, accurate tissue detection, and to provide resultant feedback to the operator to assist in the avoidance of nerve tissue injury.

### Biomarkers

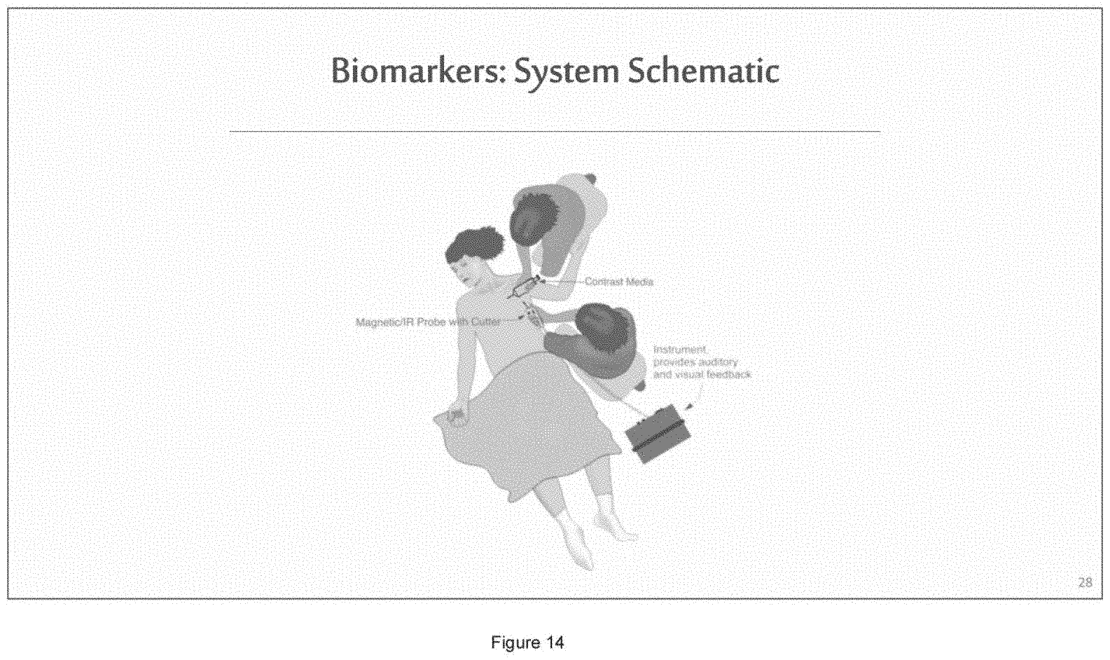

FIG. 14 shows a patient during delivery of a biomarker to better elucidate the surgical environment to directly locate anatomical features.

Biomarker tools are generally in the form of an injectable or lavage suspension of particles that attach to specific tissue in the body. The particles contain a label such as a magnetic or fluorescent compound, which is detected intraoperatively to locate target tissue.

As an example, nanoparticles with neurotargeting features such as cell surface receptor ligands have been used to label nerves.

These embodiments of the electrocautery device and system of the present invention may use biomarkers to permit direct imaging of nerve and nerve tissues.

Biomarker embodiments of the present invention permit the leveraging of axillary vein and artery imaging and identification to target nerve and nerve tissues, such as the long thoracic nerve.

Biomarker suspension may be administered systemically with intravenous infusion approximately 24 hours prior to surgery. Alternatively, a biomarker suspension may be administered locally with a vascular catheter approximately 2 hours prior to surgery.

The biomarker signal may be detected with magnetic particle imaging (MPI) or near infrared imaging (NIR), and such techniques may be used for real-time location of nerves and nerve tissues.

Nerve size and location may be irrelevant with regard to magnetic and fluorescent markers. Typically, vasculature follows nerves for efficient circulatory delivery to nerves. Biomarker sensors are normally sufficiently sensitive and selective to pick up the emitted signals.

Accordingly, in these embodiments of the electrocautery device and system of the present invention, biomarker detection and associated signaling may be incorporated in order to provide real time, accurate tissue detection, and to provide resultant feedback to the operator to assist in the avoidance of nerve tissue injury.

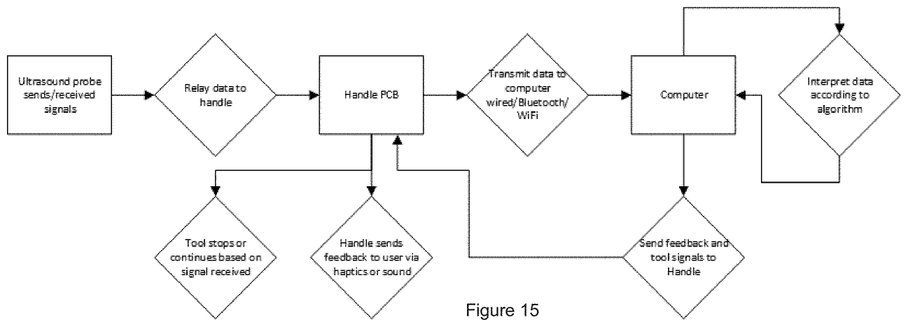

FIG. 15 is schematic of the device sensing, signal and microprocessor logic and feedback logic used in an electrocautery device and system in accordance with one embodiment of the present invention.

From this schematic, it will be appreciated that the various ultrasound data, neuromonitoring electrical stimulus, and biomarker signals, as well as three-dimensional spatial detection data, may provide visual and tactile feedback to and through the computer system to the electrocautery device in its many embodiments.

It will be appreciated that the mechanical and electromechanical arrangements in the device, the nature and distribution of the associated software, and the logical order of the steps in the described methods are used for purposes of illustration only, and that the device and the method steps may be varied where not otherwise inconsistent with the purpose and result obtained in the practice of the invention.

It will be also be appreciated that the mechanical and electromechanical arrangements in the device and the nature and distribution of the associated software within the electrocautery device and system of the present invention may be varied, as may be their individual subassemblies and elements thereof, and the steps of the method may include individual steps and series of steps within subroutines of the methods as described.

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description of which the claims are to be read as a portion thereof, and all changes that come within the meaning and range of equivalency of the claims are intended to be embraced therein.

The present invention may be used in accordance with other systems and devices relating to electrocautery surgery or surgery without electrocautery cutting, such as those described in the following references that are hereby incorporated herein by reference:

### REFERENCES

U.S. Pat. No. 10,314,642 Electrocautery method and apparatus
U.S. Pat. No. 9,339,323 Electrocautery method and apparatus
U.S. Pat. No. 8,728,072 Electrocautery method and apparatus
U.S. Pat. No. 8,696,662 Electrocautery method and apparatus
U.S. Pat. No. 7,803,156 Method and apparatus for surgical electrocautery
U.S. Pat. No. 7,794,461 Method and apparatus for surgical electrocautery
U.S. Pat. No. 6,899,712 Surgical instruments with integrated electrocautery
U.S. Pat. No. 6,451,017 Surgical instruments with integrated electrocautery
U.S. Pat. No. 6,063,081 Fluid-assisted electrocautery device
   Electrocautery Device with Electrocautery Unit and Ultrasonic Transponder

What is claimed is:

1. An electrocautery device, the device comprising:
(a) a handle portion; (b) an electrocautery unit integrated into said handle portion, and comprising a surgical electrode extending from said distal end thereof; (c) an ultrasonic transponder integrated into said handle portion and extending from said distal end thereof and disposed adjacent to said surgical electrode and so as to be adapted to detect differences in anatomical tissue types distally of said surgical electrode; (d) a source of current to said surgical electrode; (e) a feedback module adapted to either (1) signal the user of the electrocautery device or (2) change the current provided by said source of current; and (f) data transmission module for transmitting data from said ultrasonic transponder, and for transmitting data to said feedback module.

2. The electrocautery device of claim 1, additionally comprising: (g) an accelerometer and a short-range wireless or wired data transmission module adapted to transmit position and acceleration data from the electrocautery unit, as well as to transmit electrocautery unit status data.

3. The electrocautery device of claim 2, wherein said data transmission module also contains a microprocessor and RAM/ROM memory for managing the short-range wireless or wired interface and converting voltage data from the accelerometer into digitized data.

4. The electrocautery device of claim 1, additionally comprising: (h) a three-dimensional spatial data module adapted to transmit three-dimensional spatial data to a computer system.

5. The electrocautery device of claim 1, additionally comprising a system that includes a microprocessor and display adapted to display the surgical site and further incorporate position and acceleration data from the electrocautery unit.

6. The electrocautery device of claim 4, additionally comprising: (h) a three-dimensional spatial detection system and associated tactile feedback system contained within the electrocautery device and the associated computer system.
Electrocautery Device with Electrocautery Unit, Ultrasonic Transponder and Accelerometer and a Short-Range Wireless or wired Data Transmission Module

7. An electrocautery device, the device comprising:
(a) a handle portion; (b) an electrocautery unit integrated into said handle portion, and comprising a surgical electrode extending from said distal end thereof; (c) an ultrasonic transponder integrated into said handle portion and extending from said distal end thereof and disposed adjacent to said surgical electrode and so as to be adapted to detect differences in anatomical tissue types distally of said surgical electrode; (d) a source of current to said surgical electrode; (e) a feedback module adapted to either (1) signal the user of the electrocautery device or (2) change the current provided by said source of current; (f) data transmission module for transmitting data from said ultrasonic transponder, and for transmitting data to said feedback module; and (g) an accelerometer and a short-range wireless or wired data transmission module adapted to transmit position and acceleration data from the electrocautery unit, as well as to transmit electrocautery unit status data.

8. The electrocautery device of claim 7, wherein said (f) data transmission module also contains a microprocessor and RAM/ROM memory for managing the short-range wireless or wired interface and converting voltage data from the accelerometer into digitized data.

9. The electrocautery device of claim 7, additionally comprising: (h) a three-dimensional spatial data module adapted to transmit three-dimensional spatial data to a computer system.

10. The electrocautery device of claim 7, additionally comprising a system that includes (i) a microprocessor and display adapted to display the surgical site and further incorporate position and acceleration data from the electrocautery unit.

11. The electrocautery device of claim 10, additionally comprising: (h) a three-dimensional spatial detection system and associated tactile feedback system contained within the electrocautery device and the associated computer system.
Electrocautery System with Electrocautery Unit and Ultrasonic Transponder and Accelerometer and a Short-Range Wireless or Wired Data Transmission Module and Three-Dimensional Spatial Data Module

12. An electrocautery system and device, the system and device comprising:
(a) a handle portion; (b) an electrocautery unit integrated into said handle portion, and comprising a surgical electrode extending from said distal end thereof; (c) an ultrasonic transponder integrated into said handle portion and extending from said distal end thereof and disposed adjacent to said surgical electrode and so as to be adapted to detect differences in anatomical tissue types distally of said surgical electrode; (d) a source of current to said surgical electrode; (e) a feedback module adapted to either (1) signal the user of the electrocautery device or (2) change the current provided by said source of current; (f) data transmission module for transmitting data from said ultrasonic transponder, and for transmitting data to said feedback module; (g) a computer adapted to accept data from said ultrasonic transponder through said data transmission module and to process said data and signal said feedback module so as to cause said feedback module to either (1) signal the user of the electrocautery device or (2) change the current provided by said source of current; (h) an accelerometer and a short-range wireless or wired data transmission module adapted to transmit position and acceleration data from the electrocautery unit, as well as to transmit electrocautery unit status data; (i) a three-dimensional spatial data module adapted to transmit three-dimensional spatial data to a computer system; and (j) a microprocessor and display adapted to display the surgical site and further incorporate position and acceleration data from the electrocautery device.
Electrocautery System with Electrocautery Unit and Ultrasonic Transponder and Accelerometer and a Short-Range Wireless or Wired Data Transmission Module and Three-Dimensional Spatial Data Module

13. An electrocautery system and device, the system and device comprising:
(a) a handle portion; (b) an electrocautery unit integrated into said handle portion, and comprising a surgical electrode extending from said distal end thereof; (c) an ultrasonic transponder integrated into said handle portion and extending from said distal end thereof and disposed adjacent to said surgical electrode and so as to be adapted to detect differences in anatomical tissue types distally of said surgical electrode; (d) a source of current to said surgical electrode; (e) a feedback module adapted to either (1) signal the user of the electrocautery device or (2) change the current provided by said source of current; (f) data transmission module for transmitting data from said ultrasonic transponder, and for transmitting data to said feedback module; and (g) a computer adapted to accept data from said ultrasonic transponder through said data transmission module and to process said data and signal said feedback module so as to cause said feedback module to either (1) signal the user of the electrocautery device or (2) change the current provided by said source of current; (h) a three-dimensional spatial data module adapted to transmit three-dimensional spatial data to a computer system, and (i) a microprocessor and display adapted to display the surgical site and further incorporate position and acceleration data from the electrocautery device.
Surgical Cutting Device with Ultrasonic Transponder

14. A surgical cutting device, the device comprising:
(a) a handle portion; (b) a surgical cutting blade integrated into said handle portion; (c) an ultrasonic transponder integrated into said handle portion and extending from said proximal end thereof and disposed adjacent to said surgical cutting blade and so as to be adapted to detect differences in anatomical tissue types proximally of said surgical cutting blade; (d) a feedback module adapted to signal the user of the surgical cutting device; and (e) data transmission module for transmitting data from said ultrasonic transponder, and for transmitting data to said feedback module.

15. The surgical cutting device of claim 14, additionally comprising: (g) an accelerometer and a short-range wireless data transmission module adapted to transmit position and acceleration data from the surgical cutting device.

16. The surgical cutting device of claim 15, wherein said data transmission module also contains a microprocessor and RAM/ROM memory for managing the short-range wireless interface and converting voltage data from the accelerometer into digitized data.

17. The surgical cutting device of claim 14, additionally comprising: (h) a three-dimensional spatial data module adapted to transmit three-dimensional spatial data to a computer system.

18. The surgical cutting device of claim 14, additionally comprising a system that includes a microprocessor and display adapted to display the surgical site and further incorporate position and acceleration data from the electrocautery unit.

19. The surgical cutting device of claim 17, additionally comprising: (h) a three-dimensional spatial detection system and associated tactile feedback system contained within the electrocautery device and the associated computer system.
Electrocautery Device with Electrocautery Unit, Ultrasonic Transponder and Accelerometer and a Short-Range Wireless Data Transmission Module

20. A surgical cutting device, the device comprising:
(a) a handle portion; (b) a surgical cutting blade integrated into said handle portion; (c) an ultrasonic transponder integrated into said handle portion and extending from said proximal end thereof and disposed adjacent to said surgical cutting blade and so as to be adapted to detect differences in anatomical tissue types proximally of said surgical cutting blade; (d) a feedback module adapted to signal the user of the surgical cutting device; (e) data transmission module for transmitting data from said ultrasonic transponder, and for transmitting data to said feedback module; and (f) an accelerometer and a short-range wireless data transmission module adapted to transmit position and acceleration data from the surgical cutting device.

21. The surgical cutting device of claim 20, wherein said (f) data transmission module also contains a microprocessor and RAM/ROM memory for managing the short-range wireless interface and converting voltage data from the accelerometer into digitized data.

22. The electrocautery device of claim 20, additionally comprising: (h) a three-dimensional spatial data module adapted to transmit three-dimensional spatial data to a computer system.

23. The surgical cutting device of claim 20, additionally comprising a system that includes (i) a microprocessor and display adapted to display the surgical site and further incorporate position and acceleration data from the surgical cutting device.

24. The electrocautery device of claim 23, additionally comprising: (h) a three-dimensional spatial detection system and associated tactile feedback system contained within the surgical cutting device and the associated computer system.
Electrocautery System with Electrocautery Unit and Ultrasonic Transponder and Accelerometer and a Short-Range Wireless Data Transmission Module and Three-Dimensional Spatial Data Module

25. A surgical cutting system and device, the system and device comprising:
(a) a handle portion; (b) a surgical cutting blade integrated into said handle portion, and comprising a surgical cutting blade extending from said distal end thereof; (c) an ultrasonic transponder integrated into said handle portion and extending from said proximal end thereof and disposed adjacent to said surgical cutting blade and so as to be adapted to detect differences in anatomical tissue types proximally of said surgical cutting blade; (d) a feedback module adapted to signal the user of the surgical cutting device; (e) data transmission module for transmitting data from said ultrasonic transponder, and for transmitting data to said feedback module; (f) a computer adapted to accept data from said ultrasonic transponder through said data transmission module and to process said data and signal said feedback module so as to cause said feedback module to signal the user of the surgical cutting device; (g) an accelerometer and a short-range wireless data transmission module adapted to transmit position and acceleration data from the surgical cutting device; (h) a three-dimensional spatial data module adapted to transmit three-dimensional spatial data to a computer system; and (i) a microprocessor and display adapted to display the surgical site and further incorporate position and acceleration data from the surgical cutting device.
Electrocautery System with Electrocautery Unit and Ultrasonic Transponder and Accelerometer and a Short-Range Wireless Data Transmission Module and Three-Dimensional Spatial Data Module

26. A surgical cutting system and device, the system and device comprising:
(a) a handle portion; (b) a surgical cutting blade integrated into said handle portion; (c) an ultrasonic transponder integrated into said handle portion and extending from said distal end thereof and disposed adjacent to said surgical cutting and so as to be adapted to detect differences in anatomical tissue types proximally of said surgical cutting blade; (d) a feedback module adapted to signal the user of the surgical cutting device; (e) data transmission module for transmitting data from said ultrasonic transponder, and for transmitting data to said feedback module; (f) a computer adapted to accept data from said ultrasonic transponder through said data transmission module and to process said data and signal said feedback module so as to cause said feedback module to signal the user of the surgical cutting device; (g) a three-dimensional spatial data module adapted to transmit three-dimensional spatial data to a computer system, and (h) a microprocessor and display adapted to display the surgical site and further incorporate position and acceleration data from the surgical cutting device.
